(19) United States
(12) Patent Application Publication
(10) Pub. No.: US 2016/0082140 A1
(43) Pub. Date: Mar. 24, 2016

(54) FRAGRANT HANGING ORNAMENT

(71) Applicant: Sin-Hsiung CHEN, Tainan City (TW)

(72) Inventor: Sin-Hsiung CHEN, Tainan City (TW)

(21) Appl. No.: 14/494,209

(22) Filed: Sep. 23, 2014

(51) Int. Cl. A61L 9/12

(52) U.S. Cl. CPC A61L 9/127

(57) ABSTRACT

A fragrant hanging ornament has a shell, an absorbing element and a ring. The shell has a chamber and a cover. The cover has a positioning recess, a passage and at least one outlet. The passage is formed in the cover and is in communication with the positioning recess and the at least one outlet. The absorbing element is mounted in the positioning recess and is inserted into the chamber. The ring is mounted on the cover. The absorbing element absorbs a liquid fragrance product in the chamber, and the scent of the liquid fragrance product is diffused through the passage and out of the at least one outlet. A rope can be inserted through the ring, and the combination of the fragrant hanging ornament and the rope is taken as a necklace or a bracelet, so the fragrant hanging ornament is convenient to carry.

### BACKGROUND OF THE INVENTION

1. Field of the Invention

The present invention relates to a fragrant hanging ornament, and more particularly to a fragrant hanging ornament that contains a liquid fragrance product and diffuses the scent of the liquid fragrance product.

2. Description of Related Art

Smell is one of human's perceptive senses, and each fragrance has a different effect, such as calming, relaxation and refreshment. A conventional fragrant diffuser is usually put in an indoor space. The fragrant diffuser has a container and a diffusing element. The container has a body, a chamber and an opening. The chamber is formed in the body and is filled with a liquid fragrance product. The opening is formed in a top surface of the body and is in communication with the chamber. The diffusing element is mounted on the container. A bottom end of the diffusing element is inserted through the opening of the container and into the chamber of the container, and is sunk in the liquid fragrance product. A top end of the diffusing element is revealed out the opening of the container. The liquid fragrance product is siphoned from the bottom end of the diffusing element to the top end of the diffusing element. Therefore, the fragrant diffuser diffuses the scent of the liquid fragrance product by the diffusing element.

The volume of the fragrant diffuser is big. The weight of the container and the weight of the liquid fragrance product are heavy. The top end of the diffusing element is revealed out the opening of the container. Therefore, the fragrant diffuser is inconvenient to carry, and the fragrant diffuser is only suitable for being placed in the indoor space. Thus, the scent of the liquid fragrance product diffused by the fragrant diffuser is kept in the indoor space, and users cannot smell the scent of the liquid fragrance product in the outdoors.

To overcome the shortcomings, the present invention tends to provide a fragrant hanging ornament to mitigate or obviate the aforementioned problems.

### SUMMARY OF THE INVENTION

The main objective of the invention is to provide a fragrant hanging ornament that is convenient to carry.

The fragrant hanging ornament has a shell, an absorbing element and a ring. The shell has a seat, a chamber, a cover and a washer. The chamber is formed in the seat. The cover is mounted on the seat and has a positioning recess, a passage and at least one outlet. The positioning recess is formed on a bottom surface of the cover. The passage is formed in the cover and is in communication with the positioning recess. The at least one outlet is formed in an outside surface and each outlet is in communication with the passage. The washer is mounted on the cover and connected to the seat. The absorbing element is mounted in the shell and has a top end and a bottom end. The top end is mounted in the positioning recess of the cover. The bottom end is opposite to the top end and is inserted into the chamber of the seat. The ring is mounted on the cover of the shell.

A liquid fragrance product is filled into the chamber of the seat. The cover is mounted on the seat, and the washer is connected to the cover and the seat for preventing the liquid fragrance product from permeating out of the seat. The absorbing element is inserted into the liquid fragrance product in the chamber and absorbs the liquid fragrance product. The scent of the liquid fragrance product is diffused through the passage of the cover and out of the at least one outlet of the cover. A rope can be inserted through the ring, and then the combination of the fragrant hanging ornament and the rope is taken as a necklace or a bracelet, so the fragrant hanging ornament is convenient to carry. Furthermore, the scent of the liquid fragrance product is diffused out of the shell continually, and users can smell the scent of the liquid fragrance product anywhere.

### BRIEF DESCRIPTION OF THE DRAWINGS

FIG. 1 is an exploded perspective view of a fragrant hanging ornament in accordance with the present invention;

FIG. 2 is a perspective view of the fragrant hanging ornament in FIG. 1;

FIG. 3 is a side view in partial section of the fragrant hanging ornament in FIG. 2;

FIG. 4 is an operational side view in partial section of the fragrant hanging ornament in FIG. 2, filled with the liquid fragrance product;

FIG. 5 is an operational perspective view of the fragrant hanging ornament in FIG. 1, combined with a rope to serve as a necklace; and

FIG. 6 is another operational perspective view of the fragrant hanging ornament in FIG. 1, combined with a rope to serve as a bracelet.

### DETAILED DESCRIPTION OF PREFERRED EMBODIMENT

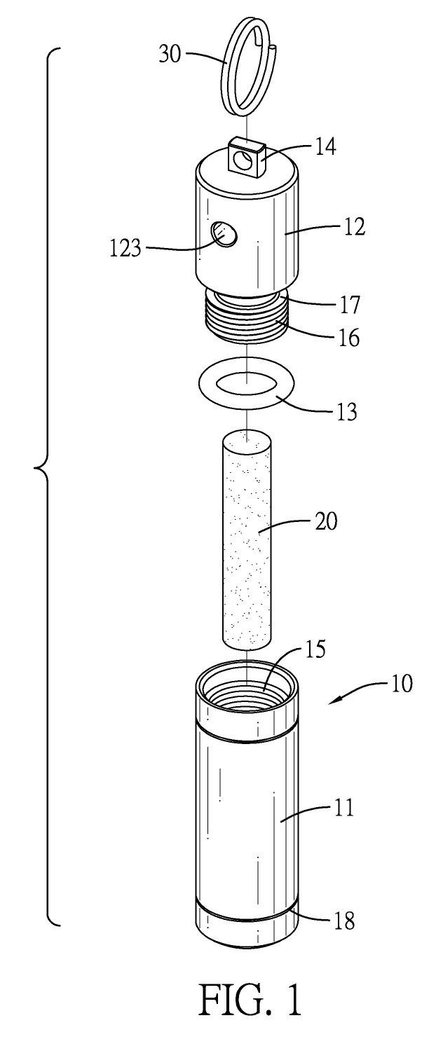

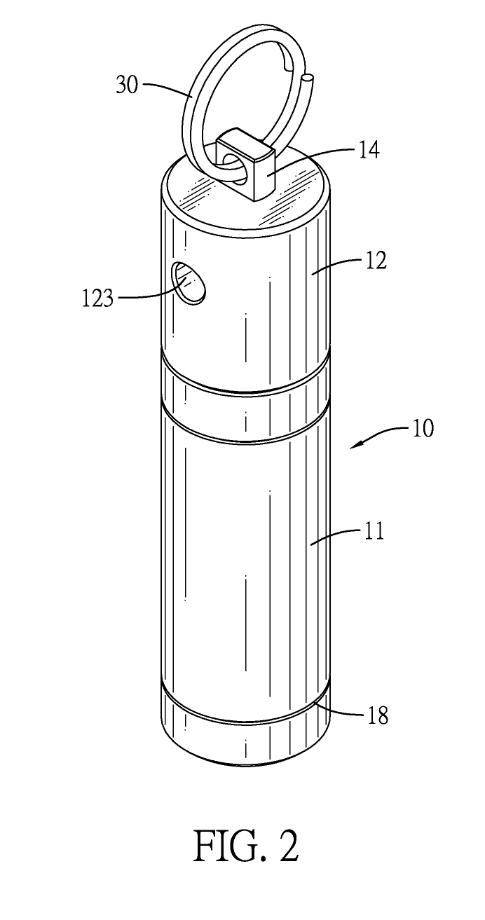

With reference to FIGS. 1 to 3, a fragrant hanging ornament in accordance with the present invention comprises a shell 10, an absorbing element 20 and a ring 30.

The shell 10 has a seat 11, a chamber 111, a cover 12 and a washer 13. The chamber 111 is formed in the seat 11. The cover 12 is mounted on the seat 11. The cover 12 has a bottom surface, an outside surface, a positioning recess 121, a passage 122 and at least one outlet 123. The positioning recess 121 is formed on the bottom surface of the cover 12. The passage 122 is formed in the cover 12 and is in communication with the positioning recess 121. The at least one outlet 123 is formed in the outside surface of the cover 12 and each outlet 123 is in communication with the passage 122. The washer 13 is mounted on the cover 12 and is connected to the seat 11. Furthermore, the cover 12 has a lifting lug 14 formed on a top surface of the cover 12.

The absorbing element 20 is mounted in the shell 10. The absorbing element 20 has a top end and a bottom end. The top end is mounted in the positioning recess 121 of the cover 12. The bottom end is opposite to the top end and is inserted into the chamber 111 of the seat 11. The absorbing element 20 may be porous.

The ring 30 is mounted on the cover 12 of the shell 10. The ring 30 is circular and is mounted on the lifting lug 14 of the cover 12.

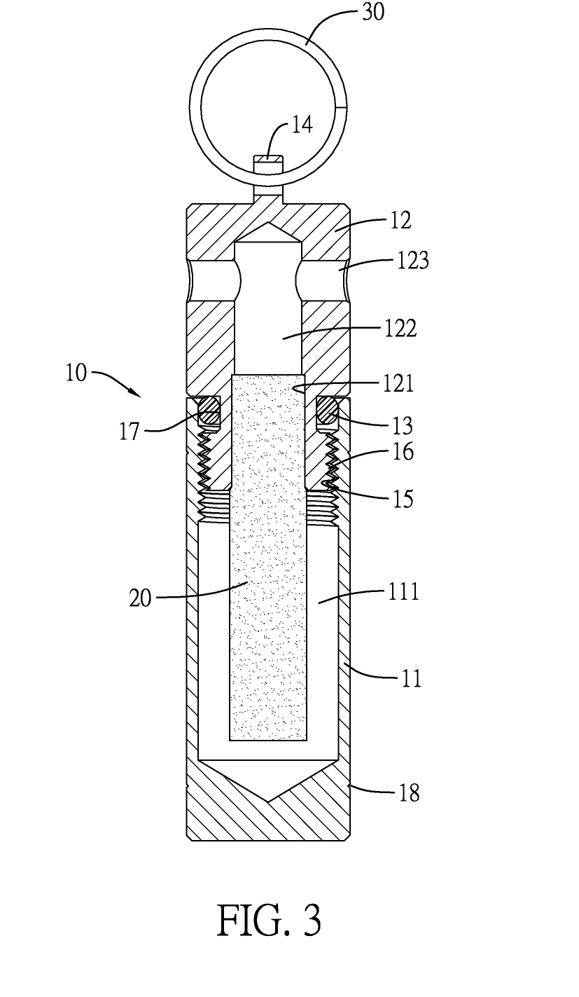

With reference to FIGS. 1 and 3, the seat 11 has an internal thread 15 and the cover 12 has an external thread 16. The internal thread 15 of the seat 11 is formed on an inside surface of the seat 11. The external thread 16 of the cover 12 is formed on the outside surface of the cover 12 and is screwed with the internal thread 15 of the seat 11. The cover 12 has an annular groove 17 formed around the outside surface of the cover 12, and the washer 13 is mounted in the annular groove 17 of the cover 12. The seat 11 has a decorative line 18 formed on an outside surface of the seat 11.

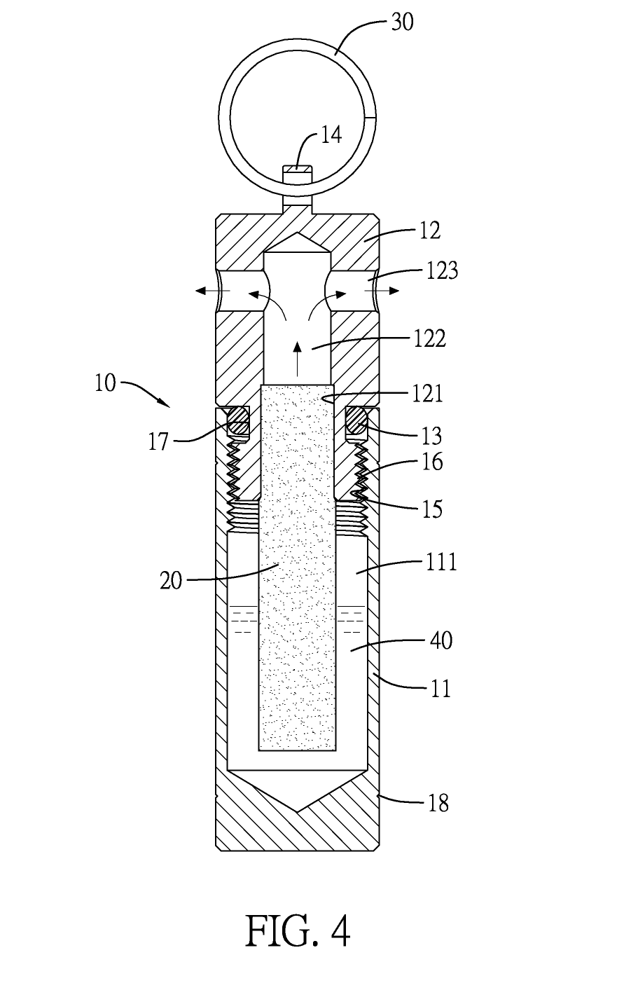

With reference to FIG. 4, a liquid fragrance product 40 is filled into the chamber 111 of the seat 11. The cover 12 is mounted on the seat 11, and the washer 13 is connected to the cover 12 and the seat 11 for preventing the liquid fragrance product in the chamber 111 from leaking out of the seat 11. The absorbing element 20 is sunk into the liquid fragrance product 40 in the chamber 111 and absorbs the liquid fragrance product 40. The scent of the liquid fragrance product 40 absorbed by the absorbing element 20 is diffused through the passage 122 of the cover 12 and out of the at least one outlet 123 of the cover 12. Thus, the scent of the liquid fragrance product 40 is diffused out of the fragrant hanging ornament under the circumstance that the cover 12 is mounted on the seat 11.

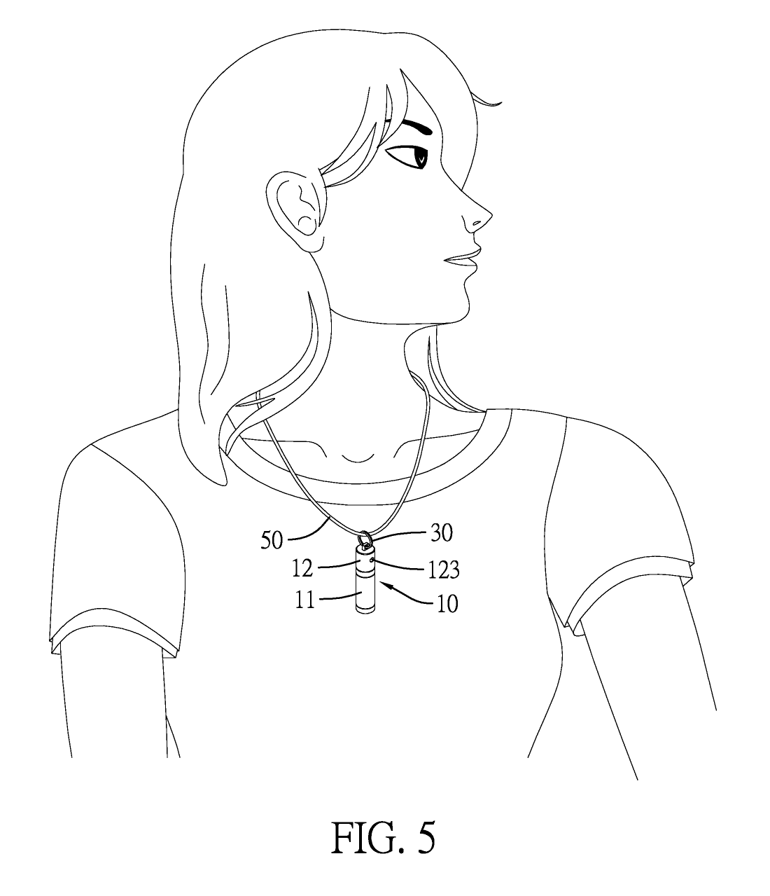

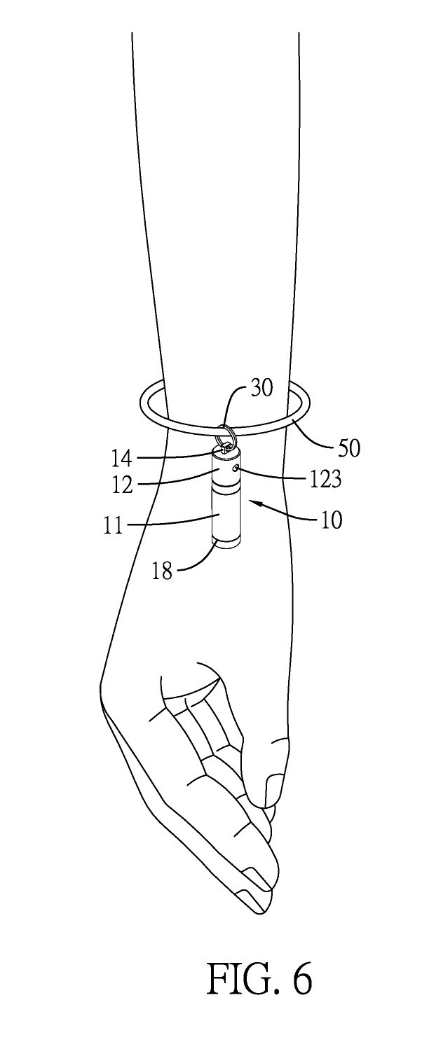

With reference to FIGS. 5 and 6, a rope 50 can be inserted through the ring 30 of the fragrant hanging ornament, and then the combination of the fragrant hanging ornament and the rope 50 is taken as a necklace or a bracelet. Therefore, the fragrant hanging ornament is convenient to carry and the scent of the liquid fragrance product 40 is diffused out of the shell 10 continually. Users can smell the scent of the liquid fragrance product 40 at any desired place. Alternatively, users can open the cover 12 to smell the scent of the liquid fragrance product 40 absorbed in the absorbing element 20 directly. Users can change a new absorbing element 20 after the fragrant hanging ornament has been used for some time.

Accordingly, the fragrant hanging ornament is convenient to carry, and the scent of the liquid fragrance product 40 absorbed in the absorbing element 20 is diffused through the passage 122 of the cover 12 and out of the at least one outlet 123 of the cover 12. Users wearing the fragrant hanging ornament can smell the scent of the liquid fragrance product 40 at any desired time and place.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

1. A fragrant hanging ornament comprising:
a shell having
a seat;
a chamber formed in the seat;
a cover mounted on the seat and having
a bottom surface;
an outside surface;
a positioning recess formed on the bottom surface of the cover;
a passage formed in the cover and communicating with the positioning recess; and
at least one outlet formed in the outside surface and each outlet communicating with the passage; and
a washer mounted on the cover and connected to the seat;
an absorbing element mounted in the shell and having
a top end mounted in the positioning recess of the cover; and
a bottom end opposite to the top end and inserted into the chamber of the seat; and
a ring mounted on the cover of the shell.

2. The fragrant hanging ornament as claimed in claim 1, wherein
the seat has
an internal thread formed on an inside surface of the seat; and
the cover has
an external thread formed on the outside surface of the cover and screwed with the internal thread of the seat.

3. The fragrant hanging ornament as claimed in claim 1, wherein the cover has an annular groove formed around the outside surface of the cover, and the washer is mounted in the annular groove of the cover.

4. The fragrant hanging ornament as claimed in claim 2, wherein the cover has an annular groove formed around the outside surface of the cover, and the washer is mounted in the annular groove of the cover.

5. The fragrant hanging ornament as claimed in claim 1, wherein the cover has a lifting lug formed on a top surface of the cover, and the ring is mounted on the lifting lug of the cover.

6. The fragrant hanging ornament as claimed in claim 2, wherein the cover has a lifting lug formed on a top surface of the cover, and the ring is mounted on the lifting lug of the cover.

7. The fragrant hanging ornament as claimed in claim 3, wherein the cover has a lifting lug formed on a top surface of the cover, and the ring is mounted on the lifting lug of the cover.

8. The fragrant hanging ornament as claimed in claim 4, wherein the cover has a lifting lug formed on a top surface of the cover, and the ring is mounted on the lifting lug of the cover.

9. The fragrant hanging ornament as claimed in claim 5, wherein the seat has a decorative line formed on an outside surface of the seat.

10. The fragrant hanging ornament as claimed in claim 6, wherein the seat has a decorative line formed on an outside surface of the seat.

11. The fragrant hanging ornament as claimed in claim 7, wherein the seat has a decorative line formed on an outside surface of the seat.

12. The fragrant hanging ornament as claimed in claim 8, wherein the seat has a decorative line formed on an outside surface of the seat.
